(19) United States
(12) Patent Application Publication
(10) Pub. No.: US 2015/0011801 A1
(43) Pub. Date: Jan. 8, 2015

(54) Catalytic Pulsed Flow Hydrogenation Of Lignin Carboxylic Acid Compounds

(71) Applicant: M. K. Carter, Lincoln, CA (US)

(72) Inventor: M. K. Carter, Lincoln, CA (US)

(73) Assignee: Carter Technologies Corporation, Lincoln, CA (US)

(21) Appl. No.: 13/936,888

(22) Filed: Jul. 8, 2013

(51) Int. Cl. C07B 31/00; C07C 29/147; C07C 1/207; C07C 37/00

(52) U.S. Cl. CPC C07B 31/00; C07C 37/002; C07C 29/147; C07C 1/2078
USPC 568/764; 568/799; 568/885; 585/639

(57) ABSTRACT

Renewable resources comprising bagasse, corn stover, wood sawdust and switch grass are subject to direct catalytic conversion or bio-fermentation producing ethanol leaving complex lignin compounds for disposal. Chemical conversion of lignin compounds (recoverable from digested lignin) to substituted phenols followed by a carbon steel catalyzed pulsed flow hydrogenation produces cresol and substituted creosol compounds. The pulsed flow process produced close to 100 percent reduction of the reactants compared to 25 percent with continuous flow and is applicable to aliphatic carboxylic acid compounds such as natural oils producing valued liquid hydrocarbons.

Specifically reactions are taught for carbon steel catalyzed pulsed flow hydrogenation of lignin carboxylic acids comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid and substituted aliphatic carboxylic acid compounds comprising citric and oleic acids in contact with a promoter comprising an anhydrous sodium sulfate and an activator comprising Co(II)-Co(III)-Co(II) using hydrogen gas at 225° C. to 350° C. and ambient to 10 atmospheres pressure.

### REFERENCES CITED

U.S. Patent Documents

| Pat. No. | Issue Date | Author | Comments |
|---|---|---|---|
| 6,429,344 | Aug. 6, 2002 | R Langer, G-M Petruck | Hydrogen partial pressure of 0.1 and 20 bar, at T of 0 to |
| | | | 140 C. with Mn dopant on noble-metal catalysts of |
| | | | subgroup VIII (Fe, Ru, Os). |
| 5,268,509 | Dec. 7, 1993 | O Immel, D Liebsch, | Catalytic hydrogenation of nitriles to form primary amines |
| | | H-H Schwarz, S Wendel, | using an iron catalyst with ammonia at 80 C. to 180 C. and |
| | | P Fischer | 20 to 400 atmospheres pressure. |
| 4,532,209 | Jul. 30, 1985 | S Hagedorn | Cresol from chemical conversion in acidic medium of |
| | | | 4-methylcyclohexa-3,5-diene-1,2-diol-1-carboxylic acid. |
| 4,431,849 | Feb. 14, 1984 | HA Colvin | Hydrogenating at 0 to 200 C. at pressure of 0 to 552 kPa for |
| | | | 0.2 to 10 hours on a catalyst of chromium, copper, |
| | | | palladium, platinum, nickel, ruthenium and rhodium. |
| 4,301,308 | Nov. 17, 1981 | R Canavesi, F Ligorati, | o-Cresol is prepared from gaseous methanol + phenol at |
| | | & G Aglietti | 200 C. to 400 C. over alumina particles. |

### BACKGROUND

1. Field of Invention

Renewable resources including bagasse, corn stover, wood sawdust, switch grass, recycled cellulose and starch materials are subject to direct catalytic conversion or bio-fermentation processes producing ethanol and organic by products leaving complex lignin compounds as waste for disposal. Chemical conversion of lignin compounds to aromatic lignin acids (recoverable from digested lignin) followed by pulsed flow hydrogenation to cresol and substituted creosol compounds prepares these natural resources for chemical conversion to a form of gasoline and industrial compounds. The pulsed flow process disclosed herein is also applicable to organic carboxylic acid compounds such as natural oils producing valued organic products and hydrocarbon fuels.

Catalytic reactions are taught for chemical hydrogenation, using a pulsed flow process, for lignin acids (recoverable from digested comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid and substituted aliphatic carboxylic acid comprising citric and oleic acid compounds in contact with a carbon steel catalyst, a promoter comprising an anhydrous sodium sulfate and an activator comprising Co(II)-Co(III)-Co(II) compound using hydrogen gas at ambient to 10 atmospheres pressure.

2. Description of Prior Art

The chemical process industry has grown to maturity based on petroleum feed stocks. Petroleum is a non-renewable resource that may become unavailable in the next 100 years. This planet Earth fosters continual growth of numerous carbohydrate based plants including fruits, vegetables and grain food sources plus their supporting plant stalks and related cellulose materials. Grains, corn cobs, the support plant stalks, trees and grasses are subject to direct catalytic conversion and bio-fermentation processes producing ethanol and organic by products leaving complex lignin compounds as waste for disposal. Chemical conversion of lignin compounds to aromatic lignin acids (recoverable from digested lignin) followed by pulsed flow hydrogenation to cresol and substituted creosol compounds prepares these natural resources for chemical conversion to a form of gasoline. A major industry is blooming in ethanol production but the published conversion efficiencies based on total cellulose starting material are low. These conversion efficiencies can be improved substantially by complete utilization of waste lignins. Ethanol is becoming more available as a renewable resource and this application teaches catalytic hydrogenation of lignin acids and non-lignin acids to valued cresols, substituted creosols and related hydrocarbons in preparation for production of a form of gasoline and chemical intermediates for use in the chemical process industry.

Prior art discloses conversion of chemical compounds derived from petroleum processes to cresols by oxidation, reactive combination or reactive ring closure but none of these reactions teach conversion of lignin acids (recoverable from digested lignin) or substituted aliphatic carboxylic acid organic compounds to cresols or aliphatic hydrocarbons respectively by a pulsed flow hydrogenation process. U.S. Pat. No. 4,301,308, issued Nov. 17, 1981, introduced a process for preparation of o-cresol by reacting methanol with vaporized phenol, an esterification reaction, at temperatures in the range of 200° C. to 400° C. over alumina particles. U.S. Pat. No. 4,431,849, issued Feb. 14, 1984, teaches a process for preparing a methyl phenol from an alkylbenzene by oxidation over a catalyst of chromium, copper, palladium, platinum, nickel, ruthenium or rhodium at 0 to 80 psi and a temperature in the range of 0° C. to 200° C. U.S. Pat. No. 4,532,209, issued Jul. 30, 1985, discloses a process for a reactive cleavage and ring closure of 4-methylcyclohexa-3,5-diene-1,2-diol-1-carboxylic acid to cresol in an acidic medium.

Nitrile compounds have been reduced to amines with hydrogen and ammonia gases on an iron catalyst at 80° C. to 180° C. and 20 to 400 atmospheres pressure as disclosed in U.S. Pat. No. 5,268,509, issued Dec. 7, 1993.

Iron, ruthenium and osmium, with a manganese dopant, have been employed as primary reaction catalysts for conversion of menthone or isomenthone or mixtures of such compounds using hydrogen gas at temperatures of 100 to 200° C. and at hydrogen partial pressures between 2 and 50 bar (30 to 750 psi) and/or by rearrangement of menthol stereoisomers in the presence of hydrogen at temperatures of 0 to 140° C. and at hydrogen partial pressures between 0.1 and 20 bar in the presence of noble-metal-containing catalysts as disclosed in U.S. Pat. No. 6,429,344, issued Aug. 6, 2002. While these are all productive catalytic hydrogenations none of these disclosures teach use of a pulsed flow process on a carbon steel catalyst for high conversion efficiency chemical reduction of carboxylic acids to methyl substituted compounds as cresols, substituted creosols, alcohols or hydrocarbon compounds.

The above reported chemical processes have been conducted using continuous flow hydrogenation of available petroleum derived chemical compounds and are, therefore, distinctly different from catalytic pulsed flow reductive hydrogenation of renewable resources, specifically lignin acid compounds, to valued cresol, substituted creosol and oxygenated gasoline products. The process disclosed herein is also applicable to organic carboxylic acid compounds known as natural fats and oils producing valued liquid hydrocarbon fuels.

### SUMMARY OF THE INVENTION

This invention describes a pulsed flow chemical hydrogenation process catalyzed by carbon steel surface, a promoter comprising an anhydrous sodium sulfate with no mineral acid or alkaline material and an activator comprising Co(II)-Co(III)-Co(II) for reduction of lignin acids (recoverable from digested lignin) and non-lignin acid organic carboxylic acid compounds to cresols, substituted creosols and hydrocarbon products using hydrogen gas at 225° C. to 350° C. and ambient to 10 atmospheres pressure. This process has been shown to be effective for reductive conversion of lignin acids (from digested lignin) comprising 3,4-dihydroxy-5-methoxybenzoic acid, 3-hydroxy-4-methoxybenzoic acid and 4-hydroxybenzoic acid as well as for aliphatic carboxylic acid compounds comprising oleic acid over carbon steel to cresols, substituted creosols and aliphatic hydrocarbons.

It is an object of this invention, therefore, to provide a catalytic process facilitating pulsed flow reductive conversion of lignin acids to cresols and creosols. It is another object of this invention to use pulsed flow hydrogenation to catalytically reduce non-lignin acid organic carboxylic acid compounds to hydrocarbons. Other objects of this invention will be apparent from the detailed description thereof which follows, and from the claims.

### DETAILED DESCRIPTION OF THE INVENTION

Catalytic pulsed flow hydrogenation of aromatic lignin acids to cresol, creosol and substituted creosol compounds prepares these valuable derivatives of natural resources for chemical conversion to a form of gasoline and valued industrial compounds. The process is also applicable to aliphatic carboxylic acid compounds such as natural oils producing valued liquid hydrocarbon fuels. Specifically catalytic reactions are taught for pulsed flow reductive chemical hydrogenation of lignin acids (recoverable from digested lignin) comprising 4-hydroxy-3,5-dimethoxybenzoic acid, 4,5-dihydroxy-3-methoxybenzoic acid, 4-hydroxy-3-methoxybenzoic acid, 4-hydroxybenzoic acid to cresol, creosol and substituted creosols, and substituted aliphatic carboxylic acid comprising citric and oleic acid compounds are reduced to hexanol and C₁₈ hydrocarbons respectively. These reductions take place with lignin acids or aliphatic carboxylic acid compounds in contact with a carbon steel catalytic surface, a promoter comprising an anhydrous sodium sulfate with no mineral acid or alkaline material and an activator comprising Co(II)-Co(III)-Co(II) for reductive hydrogenation of lignin acids and non-lignin acid organic carboxylic acid compounds to cresols, substituted creosols and hydrocarbon products using hydrogen gas at 225° C. to 350° C. and ambient to 10 atmospheres pressure.

This process employs transition metal activators for which the transition metals and directly attached atoms possess C₄ᵥ, D₄ₕ or D_{2d} point group symmetry. The activators have been designed based on a formal theory and the activators have been produced, and tested without pre-conditioning to prove their activity as prepared. The theory rests upon a requirement that activators possess a molecular string such that transitions from one molecular electronic configuration to another are barrier free so reactants may proceed freely to products as driven by thermodynamic considerations. Activators effective for stated chemical conversions to products can be made from tri-metal compounds of mixed valence produced from cobalt. These activators are made in the absence of oxygen so as to produce compounds wherein the oxidation state of the transition metal is low, typically divalent and trivalent metals. Mixed transition metal compounds have also been found to be effective activators for non-oxidative chemical conversions.

Carbon steel surfaces are the sites of catalytic hydrogenation but a promoter and an activator are required to enable the reductive chemistry. It is believed that the activator assists in bond orientation and the promoter functions to assist in bond opening. It is also apparent that water vapor, a byproduct of the reduction reaction, inhibits the rate of the reaction. Thus, by instituting a pulsed hydrogen gas flow reaction products can be swept from the steel surface with the byproduct water vapor. For example, reduction of 4-hydroxybenzoic acid with a steady gas flow produced approximately 25 percent product while the pulsed flow process produced nearly 100 percent conversion during comparable reaction times.

Thermodynamic considerations determine which chemical compounds are reduced, however reduction becomes increasingly favored as hydrogen pressure is increased. For example, 4-hydroxybenzoic acid was converted to 13 percent product at ambient hydrogen pressure while the reduction process produced nearly 100 percent product at 30 psig. Similar relative pressure related conversion efficiencies were observed for oleic acid. Thus, reductive chemical conversion of carboxylic acid compounds using a promoter on carbon steel catalytic surfaces, are taught herein producing methyl substituted analogs of the original carboxylic acid compounds.

### Activator Preparation

### Example 1

Preparation of the Co(II)-Co(III)-Co(II) activator was conducted in a short time sequence preferably in an inert gas environment.

Glass vial a—To 0.0115 g tetrachlorocatechol was added 0.0025 g Na₂CO₃ in 1 g water, heated and stirred until dissolved. Immediately was added 0.0110 g CoCl₂-6H₂O and stirred to form product A. This was heated at 160° C. for approximately 2 minutes to form product. Glass vial b—To 0.0115 g tetrachlorocatechol was added 0.0025 g Na₂CO₃ in 1 g water, heated and stirred as before until dissolved. To this was added 0.0124 g Co(NH₃)₆Cl₃ and stirred. The vial was heated at 160° C. for approximately 2 minutes to form product. Product a was mixed with product b, added an additional 1 g water and added 0.0115 g tetrachlorocatechol, heated as before and stirred until a dark color product formed. This produces the molecular string type compound identified as Co(ID-Co(III)-Co(II) (a string of three associated cobalt atoms) of mixed valence. Specifically the molecular formula for the compound is Co(II)(C₆Cl₄O₂)₂C₆Cl₄(OH)₂—Co(III)(C₆Cl₄O₂)₃—Co(II)(C₆Cl₄O₂)₂C₆Cl₄(OH)₂.

### Examples of Catalytic Chemical Conversion

Specific examples of the conditions of catalytic reductive chemical conversion to products are provided here.

### Example A

### p-Cresol Formation

The reaction equipment consisted of a 250 mL three neck round bottom pyrex glass flask fit with a thermocouple, a one eighth inch diameter stainless steel line for hydrogen gas introduction, a one quarter inch line for product vapor removal in series with a gas vent line. The reactor was wrapped with a thick layer of fiber mat insulation to maintain a uniform temperature throughout the reaction chamber. Two pieces of carbon steel, each 2″×¾″×0.032″ were placed in the bottom of the flask. The reactants, 4.0 g of 4-hydroxy benzoic acid plus 0.022 g Co(II)-Co(III)-Co(II) tetrachlorocatechol activator plus 0.405 g Na₂SO₄ promoter, were ground together in a mortar and pestle and placed in the flask on top of the steel strips. Hydrogen gas was introduced into the bottom of the flask at a flow rate of 10 mL/minute to flush air from the reactor. After flushing the reactor was heated to 285° C. to 288° C. for a period of one hour. The reactor was flushed with a short burst of hydrogen, by sharp pressure drops followed by re-pressurization, every 5 to 10 minutes to sweep out water vapor. Once the rector was cool it was opened to recover 0.41 gram (13 percent) p-cresol product (verified by boiling point).

### Example B

### p-Cresol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 3.246 g of 4-hydroxy benzoic acid plus 0.0108 g Co(II)-Co(III)-Co(II) tetrachlorocatechol activator plus 0.304 g Na₂SO₄ promoter, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas then heated to 288° C. to 290° C. for a period of three hours and forty minutes. The reactor was flushed with a short burst of hydrogen, by sharp pressure drops followed by re-pressurization, every 5 to 10 minutes to sweep out water vapor. Once the reactor was cool it was opened and 2.301 g (95.7%) crude liquid p-cresol was recovered.

### Example C

### Methoxy Cresol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 2.853 g of 4-hydroxy-3-methoxybenzoic acid plus 0.0158 g Co(II)-Co(III)-Co(II) tetrachlorocatechol activator plus 0.315 g Na₂SO₄ promoter, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 315° C. to 330° C. for a period of two hours and fifteen minutes. The reactor was flushed with a short burst of hydrogen, by sharp pressure drops followed by re-pressurization, every 5 to 10 minutes to sweep out water vapor. Once the reactor was cool it was opened and 1.31 g (57%) crude liquid methoxy cresol was recovered.

### Example D

### Dimethoxy Cresol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 3.013 g of syringic acid (4-hydroxy-3,5-dimethoxybenzoic acid) plus 0.0120 g Co(II)-Co(III)-Co(II) tetrachlorocatechol activator plus 0.356 g Na₂SO₄ promoter, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 320° C. to 345° C. for a period of two hours and fifteen minutes. The reactor was flushed with a short burst of hydrogen, by sharp pressure drops followed by re-pressurization, every 5 to 10 minutes to sweep out water vapor. Once the reactor was cool it was opened and 1.334 g (53%) crude liquid dimethoxy cresol was recovered.

### Example E

### Hexanol Formation

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 3.136 g of citric acid plus 0.0316 g Co(II)-Co(III)-Co(II) tetrachlorocatechol activator plus 0.377 g Na₂SO₄promoter, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 228° C. to 249° C. for a period of two hours. The reactor was flushed with a short burst of hydrogen, by sharp pressure drops followed by re-pressurization, every 5 to 10 minutes to sweep out water vapor. Once the reactor was cool it was opened and 0.644 g (39.5%) crude hexanol was recovered.

### Example F

### A C₁₈ Wax

The reaction equipment consisted of a 6″ long×2″ diameter steel reactor fit with a thermocouple, a one eighth inch diameter stainless steel line for hydrogen gas introduction, a one eighth inch line for product vapor removal in series with a gas vent line. The reactor was wrapped with a thick layer of fiber mat insulation to maintain a uniform temperature throughout the reaction chamber. One piece of carbon steel, each 2″×¾″×0.032″ plus the ground reactants, 5.0 g oleic acid liquid with 0.053 g Co(II)-Co(III)-Co(II) tetrachlorocatechol activator plus 0.52 g Na₂SO₄ promoter, were placed in a 30 mL glass vial that was set into the vertical reactor and the reactor top was sealed closed. Hydrogen gas was introduced into the reactor at a flow rate of 10 mL/minute to flush air from the reactor. After flushing the reactor was pressurized to 30 psig with hydrogen gas heated to 228° C. to 249° C. for a period of two hours. The reactor was flushed with a short burst of hydrogen, by sharp pressure drops followed by re-pressurization, every 5 to 10 minutes to sweep out water vapor. Once the reactor was cool it was opened and 0.13 g brown wax, octadecaneoctadecene, (10%) was recovered.

What is claimed:

1. A catalytic, pulsed flow hydrogenation process for substituted carboxylic acid compounds in contact with a carbon steel catalytic surface, a promoter comprising an anhydrous sodium sulfate with no mineral acid or alkaline material and an activator comprising Co(II)-Co(III)-Co(II) made from tri-metal compounds of mixed valence produced from cobalt using hydrogen gas at 225° C. to 350° C. and ambient to 10 atmospheres pressure forming substituted methyl compounds.

2. A catalytic, pulsed flow hydrogenation process for substituted lignin acids (recoverable from digested lignin) comprising 4-hydroxy-3,5-dimethoxybenzoic acid, 4,5-dihydroxy-3-methoxybenzoic acid, 4-hydroxy-3-methoxybenzoic acid or 4-hydroxybenzoic acid compounds in contact with a carbon steel catalytic surface, a promoter comprising an anhydrous sodium sulfate with no mineral acid or alkaline material and an activator comprising Co(II)-Co(III)-Co(II) made from tri-metal compounds of mixed valence produced from cobalt using hydrogen gas at 225° C. to 350° C. and ambient to 10 atmospheres pressure forming substituted methyl carboxylic acid compounds comprising cresols or substituted cresols.

3. A catalytic, pulsed flow hydrogenation process for substituted aliphatic carboxylic acid compounds comprising citric acid or oleic acid in contact with a carbon steel catalytic surface, a promoter comprising an anhydrous sodium sulfate with no mineral acid or alkaline material and an activator comprising Co(II)-Co(III)-Co(II) compound, made from tri-metal compounds of mixed valence produced from cobalt using hydrogen gas at 225° C. to 350° C. and ambient to 10 atmospheres pressure forming substituted methyl organic compounds comprising hexanol or C₁₈ hydrocarbons respectively.

4. A catalytic, pulsed flow hydrogenation process, wherein sharp pressure drops are followed by re-pressurization every 5 to 10 minutes, for substituted carboxylic acid compounds in contact with a carbon steel catalytic surface, a promoter comprising an anhydrous sodium sulfate with no mineral acid or alkaline material and an activator comprising Co(II)-Co(III)-Co(II) made from tri-metal compounds of mixed valence produced from cobalt using hydrogen gas at 225° C. to 350° C. and ambient to 10 atmospheres pressure forming substituted methyl compounds.

5. A catalytic, pulsed flow hydrogenation process, wherein sharp pressure drops are followed by re-pressurization every 5 to 10 minutes, for substituted lignin acids (recoverable from digested lignin) comprising 4-hydroxy-3,5-dimethoxybenzoic acid, 4,5-dihydroxy-3-methoxybenzoic acid, 4-hydroxy-3-methoxybenzoic acid or 4-hydroxybenzoic acid compounds in contact with a carbon steel catalytic surface, a promoter comprising an anhydrous sodium sulfate with no mineral acid or alkaline material and an activator comprising Co(II)-Co(III)-Co(II) made from tri-metal compounds of mixed valence produced from cobalt using hydrogen gas at 225° C. to 350° C. and ambient to 10 atmospheres pressure forming substituted methyl carboxylic acid compounds comprising cresols or substituted cresols.

6. A catalytic, pulsed flow hydrogenation process, wherein sharp pressure drops are followed by re-pressurization every 5 to 10 minutes, for substituted aliphatic carboxylic acid compounds comprising citric acid or oleic acid in contact with a carbon steel catalytic surface, a promoter comprising an anhydrous sodium sulfate with no mineral acid or alkaline material and an activator comprising Co(II)-Co(III)-Co(II) compound, made from tri-metal compounds of mixed valence produced from cobalt using hydrogen gas at 225° C. to 350° C. and ambient to 10 atmospheres pressure forming substituted methyl organic compounds comprising hexanol or C₁₈ hydrocarbons respectively.
